(19) United States
(12) Patent Application Publication
(10) Pub. No.: US 2014/0348787 A1
(43) Pub. Date: Nov. 27, 2014

(54) Methods and Compositions for Treating Ear Infections

(71) Applicant: Chris V. Simmons, Sugar Land, TX (US)

(72) Inventor: Chris V. Simmons, Sugar Land, TX (US)

(73) Assignee: PROFESSIONAL COMPOUNDING CENTERS OF AMERICA, Houston, TX (US)

(21) Appl. No.: 13/900,323

(22) Filed: May 22, 2013

(51) Int. Cl. A61K 47/34; A61K 31/58; A61K 45/06; A61K 31/496

(52) U.S. Cl. CPC A61K 47/34; A61K 31/496; A61K 31/58; A61K 45/06
USPC 424/85.4; 514/772; 514/253.08; 514/254.05; 514/174

(57) ABSTRACT

Compositions and methods for treating ear infections are disclosed. More specifically, these methods may refer to treatment of internal otitis using an otic composition, such as otic gel for animals and humans. Poloxamer otic gel may be in liquid state at room temperature and may change to gel at about temperature (64° F.) as poloxamer otic gel reaches body temperature, because of the thermo-reversible properties. Consequently, poloxamer otic gel may be an effective treatment in animals and humans, where poloxamer otic gel may reach otitis affected site and remain there for a longer period of time. Additionally, poloxamer otic gel may include APIs such as antifungals, antibiotics and corticosteroids, among others. Furthermore, poloxamer otic gel may be instilled inside the ear, employing calibrated delivery device into the vertical ear canal in order to reach the horizontal ear canal, without puncturing the tympanic membrane.

### BACKGROUND

1. Field of the Disclosure

The present disclosure relates generally to ear diseases, and more particularly, to methods and compositions for treating otitis.

2. Background

Most ear infections are characterized by inflammation. In general, this condition, referred to as “otitis”, is treated upon diagnosis to reduce the risk of conditions such as hearing loss, tinnitus, facial nerve palsy, mastoiditis, labyrinthitis, vertigo, and encephalitis. The three most common types of ear infections are otitis media, otitis interna (also known as an inner ear infection or labyrinthitis), and otitis externa (also known as an outer ear infection or swimmer's ear). The most common of these types of ear infections is otitis media.

Moreover, one of the most significant improvements in the management of chronic otitis over the past 20 years are different kinds of otic applications such as topical ointment, spray, and drops, among others, which may be applied during a suitable period of time. Current topical otitis medications may treat the problem; otic medications may include components such as glucocorticoids (corticosteroids), antibiotics, antifungals, antiparasitics, and anaesthetics, in any suitable vehicle.

However, pharmaceutical otic preparations including oil-based or aqueous vehicles may result inefficient for animal treatment, for instance, animals such as dogs may shake, rub, and scratch their ears and the otic preparation applied may be removed from the ear.

Therefore, there is a need for suitable pharmaceutical otic composition that may allow APIs to reach the affected ear site and remain (for a suitable period of time) in affected ear site.

### SUMMARY

According to various embodiments, the present disclosure relates to compositions and methods for the treatment of otitis in animals and humans. More specifically, the present disclosure refers to the application of an otic pharmaceutical composition, such as otic gel that may be applied in the internal ear of animals and humans which may enable an effective administration of a specific API, thus improving treatment outcomes. The otic gel may include about 20% to about 30% of poloxamer 407 (as a vehicle), with a variety of active pharmaceutical ingredients (API).

According to the present disclosure most stable vehicle for otic gel may be poloxamer 407. Poloxamer 407 may be used with any suitable API such as antibiotics, antifungals, corticosteroids, and antiparasitics, among others. According to some embodiments, most suitable APIs included on the otic gel may be enrofloxacin, ketoconazole, and triamcinolone, among others. Additionally, poloxamer 407 may be included in poloxamer gel by itself or in combination with poloxamer L-64.

According to one embodiment, the otic gel may be a liquid at room temperature and may be converted into a gel at temperatures of about 64° F. to 85° F. Therefore, the poloxamer otic gel may be applied in the ear as a liquid composition, and may be converted into a gel as it reaches body temperature. The poloxamer otic gel may reach the affected site and remain there for a long period of time, therefore maintaining the otic gel at the desired site for a longer period of time. The otic gel may be applied with suitable dosage taking into account different factors such as weight and species as well as the grade of infection. The otic gel may be able to spread and fill the ear canals (vertical and horizontal) reducing the recurrence of infection.

Numerous other aspects, features and advantages of the present disclosure may be made apparent from the following detailed description taken together with the drawing figures.

### BRIEF DESCRIPTION OF THE DRAWINGS

The present disclosure can be better understood by referring to the following figures. The components in the figures are not necessarily to scale, emphasis instead being placed upon illustrating the principles of the invention. In the figures, reference numerals designate corresponding parts throughout the different views.

FIG. 1 depicts a view of a canine ear anatomy, according to an embodiment.

FIG. 2 is an otic gel components block diagram, according to an embodiment.

FIG. 3 depicts a view of an internal ear infection area and the poloxamer otic gel application, according to an embodiment.

### DETAILED DESCRIPTION

The present disclosure is hereby described in detail with reference to embodiments illustrated in the drawings, which form a part hereof. In the drawings, which are not necessarily to scale or to proportion, similar symbols typically identify similar components, unless context dictates otherwise. Other embodiments may be used and/or and other changes may be made without departing from the spirit or scope of the present disclosure. The illustrative embodiments described in the detailed description are not meant to be limiting of the subject matter presented herein.

“Otic Gel” refers to a colloid substance that may be applied internally and externally to the ear for medical or any suitable purpose.

“Poloxamer 407” refers to a non-ionic surfactant (copolymers) which may be used primarily as a thickening agent and gel former, but also as a co-emulsifier, solubilizer, and consistency enhancer in creams and liquid emulsions.

“Enrofloxacin” refers to a broad-spectrum bactericidal antibiotic which may be very effective for difficult-to-treat infections, particularly those that need long-term antibiotics such as osteomyelitis, sinus infections, and otitis, among others.

“Ketoconazole” refers to the only member of imidazoles that is currently used to treat systemic skin infections and is market available as an anti-fungal.

“Triamcinolone” refers to a long-acting synthetic corticosteroid which may be used to treat several different medical conditions such as chronic inflammatory skin disease or infection-induced eczema in fungal skin infections, among others.

“Vehicles” refers to carrier materials suitable for transdermal or topical drug administration.

“Otitis” refers to an ear inflammation (internal or external) as result of an infection which may affect humans and animals.

“Treating” and “treatment” refers to a reduction in severity and/or frequency of symptoms, elimination of symptoms and/or underlying cause, prevention of the occurrence of symptoms and/or their underlying cause, and improvement or remediation of damage.

“Active pharmaceutical ingredient” refers to a chemical compound that induces a desired pharmacological, physiological effect, and include agents that are therapeutically effective, prophylactically effective, or cosmeceutical effective.

### DESCRIPTION

Canine Ear Anatomy (Prior Art)

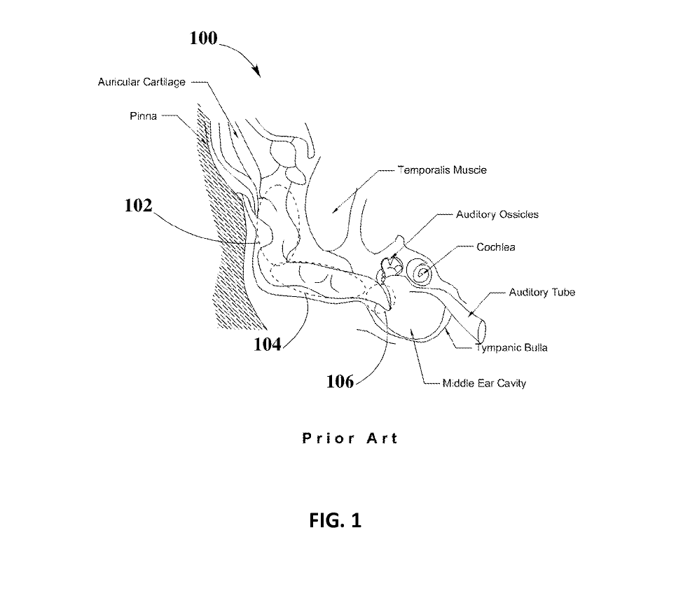

FIG. 1 depicts a view of canine ear anatomy 100. More specifically, canine ear anatomy 100 may include several components such as auricular cartilage, temporalis muscle, auditory ossicles, cochlea, auditory tube, and tympanic bulla, among others. However, according to some embodiments, the present disclosure may be focused on components such as vertical canal 102, horizontal canal 104, and tympanic membrane 106 which may be affected by the internal otitis and/or ear infections in animals.

Otitis is the third most common dog's disease in the United States. Canines have very unique ear anatomy and ear canals are difficult to treat because of the shape, for example the ear includes a vertical canal 102. Vertical canal 102 may take a very short turn in order to end up in a horizontal canal 104. The average volume of a dog's ear canals may be filled with about 1.5 ml. Most current otic preparations generally have a dosage of about 4 to 6 drops, or about 6 to 10 drops once or twice a day, therefore, since about 20 drops are needed to make one ml, dosages of 10 drops or less are not enough to fill vertical canal 102 and horizontal canal 104 of a dog. Consequently, there is a problem on the market to deliver the amount of otic compositions needed to treat internal otitis.

Furthermore, some animals may tend to shake out otic composition from the ear after otic composition has been delivered, consequently, the treatment may not be effective.

Otic Gel Composition

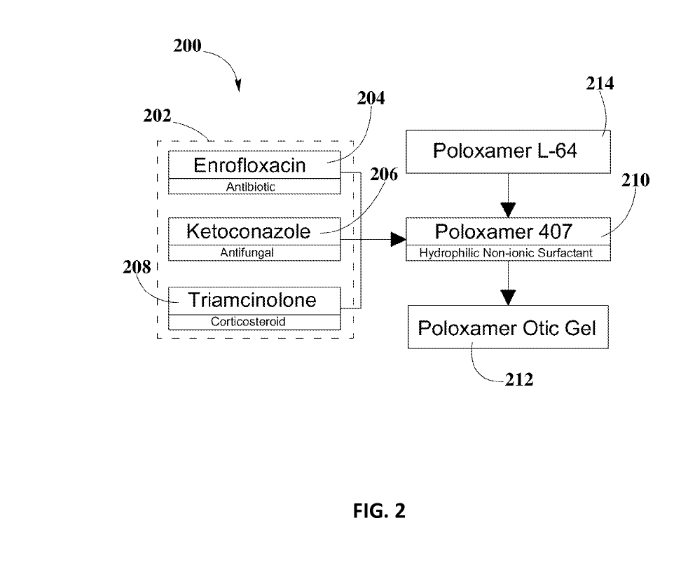

FIG. 2 depicts otic gel components block diagram 200. According to some embodiments, APIs 202 may include the active ingredients that may be used for treating otitis. APIs 202 may include antibiotics, antifungals, and corticosteroids. Suitable antibiotic may be enrofloxacin 204; otic compositions may include about 1% by weight to about 5% by weight of enrofloxacin 204. Suitable antifungal may be ketoconazole 206, otic compositions may include about 1% by weight to about 5% by weight of ketoconazole 206. Suitable corticosteroid may be triamcinolone 208, otic compositions may include about 1% by weight of triamcinolone 208. According to one embodiment, APIs 202 may be combined with a vehicle such as poloxamer 407 210. Suitable amount of poloxamer 407 210 for disclosed otic compositions may be of about 20% by weight to about 30% by weight. Poloxamer 407 210 may have thermoreversible properties, for example, poloxamer 407 210 at room temperature is in liquid state changing to a gel as poloxamer 407 210 reaches warm temperatures such as body temperature.

According to some embodiments, antibacterial agents may include amikacin, gentamicin, kanamycin, neomycin, netilmicin, streptomycin, tobramycin, paromomycin, geldanamycin, herbimycin, loracarbef, ertapenem, doripenem, imipenem, cilastatin, meropenem, cefadroxil, cefazolin, cefalotin, cefalexin, cefaclor, cefamandole, cefoxitin, defprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, teicoplanin, vancomycin, azithromycin, clarithromycin, dirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillan, bacitracin, colistin, polymyxin B, ciprofloxacin, enoxacin, gatifloxacin, levofloxacin, lomefloxacin, moxifloxacin, norfloxacin, ofloxacin, trovfloxacin, mafenide, prontosil, sulfacetamide, sulfamethizole, sulfanimilimde, sulfasalazine, sulfisoxazole, trimetoprim, demeclocycline, doxycycline, minocycline, oxytetracycline, tetracycline, arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinuspristin/dalfopristin, rifampin, tinidazole, and combinations thereof.

According to some embodiments, antifungal agents may include amrolfine, utenafine, naftifine, terbinafine, flucytosine, fluconazole, itraconazole, ketoconazole 206, posaconazole, ravuconazole, voriconazole, clotrimazole, econazole, miconazole, oxiconazole, sulconazole, terconazole, tioconazole, nikkomycin Z, caspofungin, micafungin, anidulafungin, amphotericin B, liposomal nystastin, pimaricin, griseofulvin, ciclopirox olamine, haloprogin, tolnaftate, undecylenate, clioquinol, and combinations thereof.

In further embodiments, poloxamer otic gel 212 may include other APIs 202 such as antivirals, antiparasitics, and anaesthetics, among others.

According to other embodiments, antiviral agents may include acyclovir, famciclovir and valacyclovir. Other antiviral agents include abacavir, aciclovir, adefovir, amantadine, amprenavir, arbidol., atazanavir, artipla, brivudine, cidofovir, combivir, edoxudine, efavirenz, emtricitabine, enfuvirtide, entecavir, fomvirsen, fosamprenavir, foscarnet, fosfonet, ganciclovir, gardasil, ibacitabine, immunovir, idoxuridine, imiquimod, indinavir, inosine, integrase inhibitors, interferons, including interferon type III, interferon type II, interferon type I, lamivudine, lopinavir, loviride, MK-0518, maraviroc, moroxydine, nelfinavir, nevirapine, nexavir, nucleoside analogues, oseltamivir, penciclovir, peramivir, pleconaril, podophyllotoxin, protease inhibitors, reverse transcriptase inhibitors, ribavirin, rimantadine, ritonavir, saquinavir, stavudine, tenofovir, tenofovir disoproxil, tipranavir, trifluridine, trizivir, tromantadine, truvada, valganciclovir, vicriviroc, vidarabine, viramidine, zalcitabine, zanamivir, zidovudine, and combinations thereof.

According to other embodiments, corticosteroids may include hydrocortisone, prednisone, fluprednisolone, triamcinolone, dexamethasone, betamethasone, cortisone, prednilosone, methylprednisolone, fluocinolone acetonide, flurandrenolone acetonide, and fluorometholone.

According to other embodiments, antiparasitic agents may include amitraz, amoscanate, avermectin, carbadox, diethylcarbamizine, dimetridazole, diminazene, ivermectin, macrofilaricide, malathion, mitaban, oxamniquine, permethrin, praziquantel, prantel pamoate, selamectin, sodium stibogluconate, thiabendazole, and combinations thereof.

According to other embodiments, anaesthetics agents may include benzocaine, butamben picrate, tetracaine, dibucaine, prilocalne, etidocaine, mepivacaine, bupivicaine, and lidocaine. Preferred non-steroidal anti-inflammatory agents include, for example, detoprofen, diclofenac, diflunisal, etodolac, fenoprofen, flurbiprofen, indomethacin, ketoprofen, mechlofenameate, mefenamic acid, meloxicam, nabumeone, naproxen sodium, oxaprozin, piroxicam, sulindac, tolmeting, celecoxib, rofecoxib, choline salicylate, salsate, sodium salicylate, magnesium salicylate, aspirin, ibuprofen, paracetamol, acetaminophen, and pseudoephedrine. Preferred steroids include, for example, hydrocortisone, prednisone, fluprednisolone, triamcinolone, dexamethasone, betamethasone, cortisone, prednilosone, methylprednisolone, fluocinolone acetonide, flurandrenolone acetonide, and fluorometholone, among others.

Additionally, various additives, known to those skilled in the art, may be included in poloxamer otic gel 212 to facilitate the preparation of suitable forms for patient applications. Additives may include humectants, pH adjusting agents, preservatives, emulsifiers, occlusive agents, opacifiers, antioxidants, fragrance, colorants, gelling agents, thickening agents, stabilizers, and surfactants, among others.

According to one embodiment, APIs 202 may be mixed with poloxamer 407 210, which may be previously dissolved in a suitable solvent, in order to produce poloxamer otic gel 212. Suitable solvents may be water and DMSO (dimethyl sulfoxide), among others. Poloxamer otic gel 212 may be employed for treating otitis in animals such as dogs, cats, horses, lions, among others. In other embodiments, poloxamer otic gel 212 may be used to treat otitis in humans.

Poloxamer otic gel 212, including poloxamer 407 210, may change from liquid form into gel form at temperatures of about 64° F.

In a further embodiment, poloxamer otic gel 212 may include poloxamer L-64 214 by itself or in combination with poloxamer 407 210, therefore, poloxamer otic gel 212 may be converted from liquid form to gel form at temperatures of about 85 F. Poloxamer L-64 may increase the temperature at which poloxamer otic gel 212 may be converted into a gel. Poloxamer otic gel 212 may be converted into a gel as it reaches body temperature, allowing poloxamer otic gel 212 to have a longer residence time on the affected site.

Poloxamer Otic Gel Application

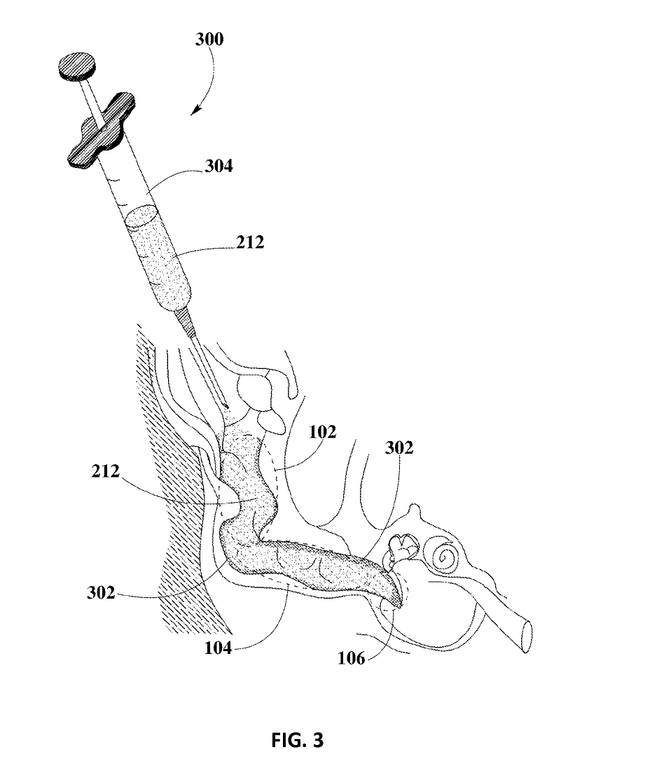

FIG. 3 depicts a view for the application of poloxamer otic gel 212 into internal ear infection area 300. Specifically, according to one embodiment, poloxamer otic gel 212 dosage may vary according to the animal size or weight, as an example, small animals may need about 0.5 ml of poloxamer otic gel 212, which may be applied using calibrated delivery device 304. Furthermore, animals over 100 pounds may need about 2 ml to 4 ml of poloxamer otic gel 212 in infected ear, however, the delivery average may be of about 1.5 ml. Before employing calibrated delivery device 304, calibrated delivery device 304 should be sterilized.

According to some embodiments, poloxamer otic gel 212 may be instilled, employing calibrated delivery device 304 through vertical canal 102 in order to reach horizontal canal 104, without puncturing tympanic membrane 106. Finally, poloxamer otic gel 212 may reach otitis 302 affected site. In one embodiment, poloxamer otic gel 212 may be instilled once, a single dose may be enough to observe healing within the next 7 days. Poloxamer otic gel 212 may be applied every week thereafter as needed.

Poloxamer otic gel 212 may stick to the walls of horizontal canal 104 and may form a hollow tube, as such; if the animal being treated shakes the head (which may be a typical reaction) poloxamer otic gel 212 may remain in the affected site. According to one embodiment, poloxamer otic gel 212 may remain in the affected site for a long period of time, thus keeping the APIs at the desired site of action where poloxamer otic gel 212 may be able to fill the ear horizontal canal 104 which may reduce recurrence of infection since all otitis 302 infected areas may be in contact with poloxamer otic gel 212.

In other embodiments, poloxamer otic gel 212 may be employed to treat otitis 302 in humans.

### Examples

Example #1 is an application of poloxamer otic gel 212, which may be used to treat otitis 302 in humans, applying a dosage of about 0.5 ml of poloxamer otic gel 212 in infected ear once a week. Healing may be achieved within 7 days, moreover, poloxamer otic gel 212 may be applied every week thereafter if needed.

While various aspects and embodiments have been disclosed here, other aspects and embodiments may be contemplated. The various aspects and embodiments disclosed here are for purposes of illustration and are not intended to be limiting, with the true scope and spirit being indicated by the following claims.

1. An otic pharmaceutical composition, comprising:
a) an active pharmaceutical ingredient (API); and
b) a vehicle, wherein the vehicle is poloxamer 407;
wherein the composition is a gel.

2. The otic pharmaceutical composition of claim 1, wherein the composition comprises 20% to 30% poloxamer 407.

3. The otic pharmaceutical composition of claim 1, wherein the vehicle further comprises poloxamer L-64.

4. The otic pharmaceutical composition of claim 1, wherein the composition is liquid at room temperature and a gel at 64° F. to 85° F.

5. The otic pharmaceutical composition of claim 1, wherein the API is selected from the group consisting of an antibacterial, antifungal, corticosteroid, antiparasitic, antiviral, anaesthetic, and non-steroidal anti-inflammatory agent.

6. The otic pharmaceutical composition of claim 5, wherein the antibacterial is an antibiotic.

7. The otic pharmaceutical composition of claim 6, wherein the antibacterial is selected from the group consisting of enrofloxacin, amikacin, gentamicin, kanamycin, neomycin, netilmicin, streptomycin, tobramycin, paromomycin, geldanamycin, herbimycin, loracarbef, ertapenem, doripenem, imipenem, cilastatin, meropenem, cefadroxil, cefazolin, cefalotin, cefalexin, cefaclor, cefamandole, cefoxitin, defprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, teicoplanin, vancomycin, azithromycin, clarithromycin, dirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillan, bacitracin, colistin, polymyxin B, ciprofloxacin, enoxacin, gatifloxacin, levofloxacin, lomefloxacin, moxifloxacin, norfloxacin, ofloxacin, trovfloxacin, mafenide, prontosil, sulfacetamide, sulfamethizole, sulfanimilimde, sulfasalazine, sulfisoxazole, trimetoprim, demeclocycline, doxycycline, minocycline, oxytetracycline, tetracycline, arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinuspristin/dalfopristin, rifampin, tinidazole, and combinations thereof.

8. The otic pharmaceutical composition of claim 7, wherein the enrofloxacin is about 1% to about 5% by weight of the composition.

9. The otic pharmaceutical composition of claim 5, wherein the antifungal is selected from the group consisting of amrolfine, utenafine, naftifine, terbinafine, flucytosine, fluconazole, itraconazole, ketoconazole, posaconazole, ravuconazole, voriconazole, clotrimazole, econazole, miconazole, oxiconazole, sulconazole, terconazole, tioconazole, nikkomycin Z, caspofungin, micafungin, anidulafungin, amphotericin B, liposomal nystastin, pimaricin, griseofulvin, ciclopirox olamine, haloprogin, tolnaftate, undecylenate, clioquinol, and combinations thereof.

10. The otic pharmaceutical composition of claim 9, wherein the ketoconazole is about 1% to about 5% by weight of the composition.

11. The otic pharmaceutical composition of claim 5, wherein the corticosteroid is selected from the group consisting of hydrocortisone, prednisone, fluprednisolone, triamcinolone, dexamethasone, betamethasone, cortisone, prednilosone, methylprednisolone, fluocinolone acetonide, flurandrenolone acetonide, and fluorometholone.

12. The otic pharmaceutical composition of claim 11, wherein the triamcinolone is about 1% by weight of the composition.

13. The otic pharmaceutical composition of claim 5, wherein the antiviral is selected from the group consisting of acyclovir, famciclovir and valacyclovir. Other antiviral agents include abacavir, aciclovir, adefovir, amantadine, amprenavir, arbidol., atazanavir, artipla, brivudine, cidofovir, combivir, edoxudine, efavirenz, emtricitabine, enfuvirtide, entecavir, fomvirsen, fosamprenavir, foscarnet, fosfonet, ganciclovir, gardasil, ibacitabine, immunovir, idoxuridine, imiquimod, indinavir, inosine, integrase inhibitors, interferons, including interferon type III, interferon type II, interferon type I, lamivudine, lopinavir, loviride, MK-0518, maraviroc, moroxydine, nelfinavir, nevirapine, nexavir, nucleoside analogues, oseltamivir, penciclovir, peramivir, pleconaril, podophyllotoxin, protease inhibitors, reverse transcriptase inhibitors, ribavirin, rimantadine, ritonavir, saquinavir, stavudine, tenofovir, tenofovir disoproxil, tipranavir, trifluridine, trizivir, tromantadine, truvada, valganciclovir, vicriviroc, vidarabine, viramidine, zalcitabine, zanamivir, zidovudine, and combinations thereof.

14. The otic pharmaceutical composition of claim 5, wherein the antiparasitic is selected from the group consisting of amitraz, amoscanate, avermectin, carbadox, diethylcarbamizine, dimetridazole, diminazene, ivermectin, macrofilaricide, malathion, mitaban, oxamniquine, permethrin, praziquantel, prantel pamoate, selamectin, sodium stibogluconate, thiabendazole, and combinations thereof.

15. The otic pharmaceutical composition of claim 5, wherein the anaesthetic is selected from the group consisting of benzocaine, butamben picrate, tetracaine, dibucaine, prilocalne, etidocaine, mepivacaine, bupivicaine, lidocaine, and combinations thereof.

16. The otic pharmaceutical composition of claim 5, wherein the non-steroidal anti-inflammatory agent is selected from the group consisting of detoprofen, diclofenac, diflunisal, etodolac, fenoprofen, flurbiprofen, indomethacin, ketoprofen, mechlofenameate, mefenamic acid, meloxicam, nabumeone, naproxen sodium, oxaprozin, piroxicam, sulindac, tolmeting, celecoxib, rofecoxib, choline salicylate, salsate, sodium salicylate, magnesium salicylate, aspirin, ibuprofen, paracetamol, acetaminophen, pseudoephedrine, and combinations thereof.

17. A method of treating otitis media or otitis interna, comprising administering an otic pharmaceutical composition to internal ear of an animal or human, wherein the composition is a gel and comprises a) an active pharmaceutical ingredient (API); and b) a vehicle, wherein the vehicle is poloxamer 407.

18. The method of claim 17, wherein the composition comprises 20% to 30% poloxamer 407.

19. The method of claim 17, wherein the vehicle further comprises poloxamer L-64.

20. The method of claim 17, wherein the composition is liquid at room temperature and a gel at 64° F. to 85° F.

21. The method of claim 17, wherein the API is selected from the group consisting of an antibacterial, antifungal, corticosteroid, antiparasitic, antiviral, anaesthetic, and non-steroidal anti-inflammatory agent.

22. The method of claim 21, wherein the antibacterial comprises enrofloxacin, the antifungal comprises ketoconazole, the corticosteroid comprises triamcinolone, the antiparasitic comprises amitraz, the anaesthetic comprises benzocaine, and the non-steroidal anti-inflammatory agent comprises detoprofen.
